(19) United States
(12) Patent Application Publication
(10) Pub. No.: US 2013/0123664 A1
(43) Pub. Date: May 16, 2013

(54) MEDICAL DEVICE HAVING A LUBRICIOUS COATING WITH A HYDROPHILIC COMPOUND IN AN INTERLOCKING NETWORK

(71) Applicant: ABBOTT CARDIOVASCULAR SYSTEMS INC., Santa Clara, CA (US)

(72) Inventors: Tung-Liang Lin, Temecula, CA (US); Jeong S. Lee, Diamond Bar, CA (US)

(73) Assignee: ABBOTT CARDIOVASCULAR SYSTEMS INC., Santa Clara, CA (US)

(21) Appl. No.: 13/736,667

(22) Filed: Jan. 8, 2013

Related U.S. Application Data
(63) Continuation-in-part of application No. 11/834,164, filed on Aug. 6, 2007.

(51) Int. Cl. A61M 25/00; A61B 5/00

(52) U.S. Cl. CPC A61M 25/0045; A61B 5/6851
USPC 600/585; 427/2.1; 427/2.3; 604/265

(57) ABSTRACT

A medical device having a lubricious coating on at least a section of the medical device, and a method of coating a medical device, the lubricious coating being a network of short chain and long chain hydrophilic compounds cross-linked to one another and interlocked with a network of a cross-linked polymerized multifunctional monomer or polymer. The coating can include one or more agents which provide enhanced adhesion of the coating on the device, or which provide faster hydration of the coating and/or improved lubricity. Additionally, the lubricious coating can be provided with one or more therapeutic or diagnostic agents, and in one embodiment the agent elutes relatively quickly in a concentrated release from the lubricious coating upon hydration of the coating.

### CROSS-REFERENCES TO RELATED APPLICATIONS

This application is a continuation-in-part of U.S. Ser. No. 11/834,164, filed Aug. 6, 2007, which is incorporated by reference in its entirety herein.

### BACKGROUND OF THE INVENTION

This invention relates to the field of lubricious hydrophilic coatings for intracorporeal medical devices such as a catheter or guidewire.

The use of a medical devices within a patient may be facilitated by the presence of a lubricious surface on the device. For example, intravascular devices, such as catheters and guidewires, are more easily maneuvered within a patient's vasculature when the friction between the walls of the vessel and the intravascular device is reduced. The friction may be reduced by coating the device with a hydrophilic compound which becomes slippery after adsorbing an appreciable amount of water. Consequently, the hydrophilic coating provides lubricity when the coated device is exposed to an aqueous solution, as when the coated device is exposed to water prior to insertion in the patient or to the patient's blood during use.

In addition to lowering the coefficient of friction of the coated device, an effective lubricious coating must strongly adhere to the device surface. The lubricious coating should remain adhered to the device surface during potentially extended periods of storage, as well as in response to abrasive forces encountered during use. Poor adhesive strength is undesirable because the lost coating may be left behind inside the patient during use, with a corresponding decrease in the lubricity of the device. Typically, a trade off exists between a coating's lubricity and the coating's adhesive and cohesive strength, so that attempts to increase the durability of lubricious coatings may inadvertently decrease the lubricity of the coating. Durability is particularly an issue on the surfaces of catheters and guidewires which are subjected to significant rubbing and abrasive forces as the devices are slidably advanced through the patient's tortuous vasculature. Consequently, one difficulty has been providing a highly lubricious coating with long lasting lubricity on a surface of a catheter or guidewire. An additional difficulty that has been encountered is to provide a hydrophilic coating that is both very durable as well capable of providing lubricity very quickly upon exposure to an aqueous solution.

It would be a significant advance to provide a highly durable hydrophilic coating on a surface of a medical device to render the device highly lubricious very quickly upon exposure to an aqueous solution. The present invention satisfies these and other needs.

### SUMMARY OF THE INVENTION

The invention is directed to a medical device having a lubricious coating on at least a section of the medical device, the lubricious coating comprising a network of hydrophilic compounds cross-linked to one another and interlocked with a network of a multifunctional polymerized compound. One aspect of the invention is a method of coating a medical device with the lubricious coating. Additional aspects of the invention are directed to including one or more agents in the coating which provide enhanced adhesion of the coating on the device, or which provide faster hydration of the coating and/or improved lubricity. Additionally, the lubricious coating can be provided with one or more therapeutic or diagnostic agents, and in one embodiment the agent elutes relatively quickly in a concentrated release from the lubricious coating upon hydration of the coating during use of the device.

The lubricious coating comprises the cured reaction product of a solution mixture which is applied onto a surface of the medical device and then cured on the device. The solution mixture is formed by mixing together at least the following components: a multifunctional monomer or polymer network-forming compounds, at least two hydrophilic compounds, one or more first cross-linkers for cross-linking the multifunctional monomer or polymer, and one or more second cross-linkers, different than the first cross-linkers, for cross-linking the hydrophilic compounds. The first cross-linkers preferentially cross-link the multifunctional monomer or polymer relative to the hydrophilic compounds, and the second cross-linkers preferentially cross-link the hydrophilic compounds relative to the multifunctional monomer or polymer. The hydrophilic compounds comprise a combination of a long chain hydrophilic polymer and a short chain polymer. In a presently preferred embodiment, the network-forming compound is an oligomer during preparation of the solution mixture. However, it may alternatively be added to the solution mixture as a monomer (prepolymerization) or as a longer chain polymer, such that it may undergo a greater or lesser degree of polymerization on the device depending on whether it is added as a monomer, oligomer, or longer chain polymer. Irrespective of whether or not the network-forming compound is added to the solution mixture in the form of a monomer or a relatively low or high molecular weight polymer, it should be understood that the multifunctional monomer or polymer of the solution mixture is in a polymerized state in the finished coating on the device.

The cross-linkers are preferably photo cross-linkers which initiate the cross-linking reactions in response to irradiation with light (e.g., of the ultraviolet or visible wavelengths). However, thermal initiators, such as peroxides, which respond to increased temperature could be used in an alternative embodiment. Thus, although discussed below primarily in terms of the preferred photo cross-linkers for photo-curing the coating, it should be understood that alternative embodiments may include one or more alternative initiators which react by other mechanisms. The terminology photo cross-linkers should be understood to refer to compounds that work by various mechanisms to cause the network-forming cross-linking, including cross-linking agents that become incorporated into the network, or alternatively, photoinitiators that form radicals that result in the cross-linking reaction.

Applied to the surface of a catheter, a guiding catheter (guide), or guidewire, the lubricious coating maintains its lubricity despite the significant rubbing and abrasive force encountered during use. While the long chain hydrophilic polymer of the formulation imparts a high degree of durability to lubricious coatings, the short chain hydrophilic polymer enables the coating to become hydrated very quickly so as to impart lubricity almost instantaneously upon exposure to an aqueous solution. The resulting coating would have a significant amount of the short chainhydrophilic compounds noncross-linked and only relatively weakly mechanically contained in the polymer network.

While not intending to be bound by theory, it is believed that the coating formulation of the invention allows for the hydrophilic compounds to become chemically interlocked by cross-linking to one another (via the second photo cross-linker) to form a true interpenetrating network with the cross-linked polymer, without having the cross-linked polymer chemically (covalently) bond to the hydrophilic compounds, for enhanced durability with good lubricity. Thus, it is believed that the hydrophilic compound network and the polymer network, which are chemically formed at the same time in the same mixture, are essentially permanently mechanically interlocked together. The coating is thus unlike a semi-IPN in which a noncross-linked hydrophilic compound is non-permanently mechanically intertwined/contained in a cross-linked polymer, and unlike a coating in which a matrix or underlayer polymer is used to chemically bond to the hydrophilic compound.

In one embodiment, the coating includes an adhesion promoter which improves the adhesion of the coating onto a polymeric or metal surface of the medical device. The adhesion promoter provides sufficiently strong adhesion onto the surface of the medical device, to thereby avoid the need for a reactive primer layer underneath the coating on the surface of the medical device.

A method of providing a lubricious coating for a medical device generally comprises preparing a solution mixture of a multifunctional monomer or polymer, a long chain as well as a short chain hydrophilic compound, one or more first initiators which preferentially cross-links the monomer or polymer relative to the hydrophilic compounds, and one or more second initiators, different than the first initiator, which preferentially cross-links the hydrophilic compounds relative to the monomer or polymer, and applying a coating of the solution mixture onto the surface of at least a section of the medical device. The coating of applied solution is then cured with radiation, for example, with UV light having UVC intensity of 3-15 mW/cm2 for 5˜20 seconds, such that the resulting lubricious coating is a network of the hydrophilic compounds cross-linked to one another and interlocked with a network of the polymerized multifunctional monomer or polymer.

In a presently preferred embodiment, the hydrophilic compounds are poylvinylpyrrolidones, the second photo cross-linker is a diazido compound, the multifunctional monomer or polymer is an acrylate oligomer, and the adhesion promoter is an acid functionalized acrylate. The resulting coating comprises an acrylate network of the polymerized multifunctional acrylate cross-linked to itself and to the cross-linked acid functionalized acrylate adhesion promoter, and a hydrophilic compound network of the polyvinylpyrrolidones cross-linked to one another by the diazido photo cross-linker, such that the hydrophilic compound network is interlocked with the acrylate network. The coated device can be e-beam or ethylene oxide (EtO) sterilized without significantly decreasing the lubricity or durability of the coating.

The lubricious coating of the invention provides significant and long-lasting lubricity immediately upon exposure to an aqueous solution such as blood or water. As a result, when applied to a catheter, a guide, and/or guidewire, the hydrated lubricious coating significantly reduces the frictional forces of the guidewire and the surface of a catheter and a guide shaft during advancement or retraction within a patient's body lumen for an extended period of time. These and other advantages of the invention will become more apparent from the following detailed description of the invention and the accompanying exemplary drawings.

### BRIEF DESCRIPTION OF THE DRAWINGS

FIG. 1 is an elevational view, partially in section, of a balloon catheter having a lubricious coating of the invention on the catheter shaft.

FIGS. 2, 3, and 4 are transverse cross sectional views of the catheter of FIG. 1, taken along lines 2-2, 3-3, and 4-4, respectively.

FIG. 4a is a transverse cross sectional view of an alternative embodiment, in which a catheter distal tip has the lubricious coating on an inner and outer surface of the distal tip, and has a less lubricious coating on the outer surface lubricious coating.

FIG. 5 illustrates a guidewire having a lubricious coating of the invention.

FIG. 6 is a transverse cross sectional view of the guidewire of FIG. 5, taken along line 6-6.

### DETAILED DESCRIPTION OF THE PREFERRED EMBODIMENTS

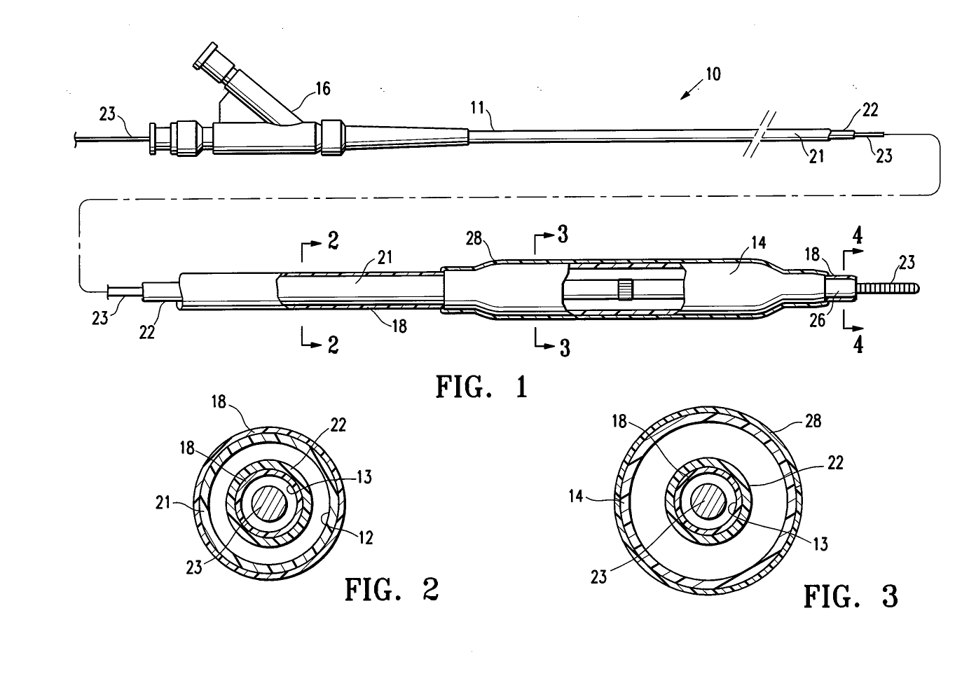

FIG. 1 illustrates one embodiment of the invention in which the medical device having a lubricious coating of the invention is a balloon catheter 10. The balloon catheter 10 generally comprises an elongated catheter shaft 11 having an inflation lumen 12 and a guidewire lumen 13 (see FIG. 2), and an inflatable balloon 14 on a distal shaft section with an interior in fluid communication with the inflation lumen. An adapter mounted 16 on the proximal end of the catheter shaft provides access to the guidewire lumen and connects to a source of inflation fluid (not shown) for inflating the balloon 14. As best shown in FIGS. 2 and 3, illustrating transverse cross sectional views of the catheter of FIG. 1 taken along lines 2-2 and 3-3, respectively, in the embodiment of FIG. 1, the shaft comprises an outer tubular member 21 having the inflation lumen 12 therein, and an inner tubular member 22 disposed in a lumen of the outer tubular member and having the guidewire lumen 13 therein configured to slidably receive a guidewire 23. The balloon 14 has a proximal skirt section sealingly secured to the distal end of the outer tubular member 21, and a distal skirt section sealingly secured to a distal end section of the inner tubular member 22, and an inflatable section therebetween. The catheter 10 can be advanced within a patient's body lumen, together with guidewire 23 or slidably advanced over previously introduced guidewire 23, to a desired location in the patient's body lumen, and the balloon 14 inflated to perform a medical procedure such as dilatation of a stenosis or expansion of a stent. When used as a stent delivery catheter, a stent 30 (see FIG. 5) is mounted on the balloon 14 for delivery and expansion within the patient's body lumen.

The catheter 10 has at least a section coated with a lubricious coating 18 of the invention, and more specifically has the lubricious coating 18 on at least a section of the shaft 11. In the embodiment of FIG. 1, the lubricious coating 18 is on the outer surface of the outer tubular member 21 (the outer lubricious coating), and on the inner surface of the inner tubular member 22 (see FIGS. 2 and 3), and on a distal tip section 26 of the shaft 11. The outer lubricious coating 18 can be provided on various lengths of the catheter 10, including on the entire outer length of the catheter from the proximal adapter 16 to the distal-most end of the distal tip section 26 (i.e., along the outer surface of the outer tubular member 21, the balloon 14, and the distal tip section 26), or on a shorter length, such that the outer lubricious coating 18 typically extends from the distal-most end of the catheter, proximally for at least about 25 to about 40 cm. For example, in one embodiment, the lubricious coating 18 extends along a 25 to 40 cm portion of the catheter along the outer surface of the distal tip section 26, the balloon 14, and only a distal portion of the outer tubular member 21. If the catheter 10 is used for delivery of a stent, a section of the balloon may be masked during coating, so that the stent can be mounted on a noncoated section of the balloon for good stent retention. The lubricious coating 18 on the inner surface of the inner tubular member may extend along the entire length of the inner tubular member 22 from the proximal to the distal end thereof, or along a shorter length. In embodiments in which the lubricious coating 18 is on the inner surface of the inner tubular member and the coating 18 is photo-cured, the inner tubular member is preferably formed of a polymer transparent to the radiation used to cross-link the coating 18. In the embodiment of FIG. 1, the outer surface of the balloon 14 has a coating 28, typically a lubricious coating, different than the lubricious coating 18 on the shaft 11, as discussed in more detail below. However, as discussed above, the balloon 14 can additionally or alternatively be coated with the lubricious coating 18.

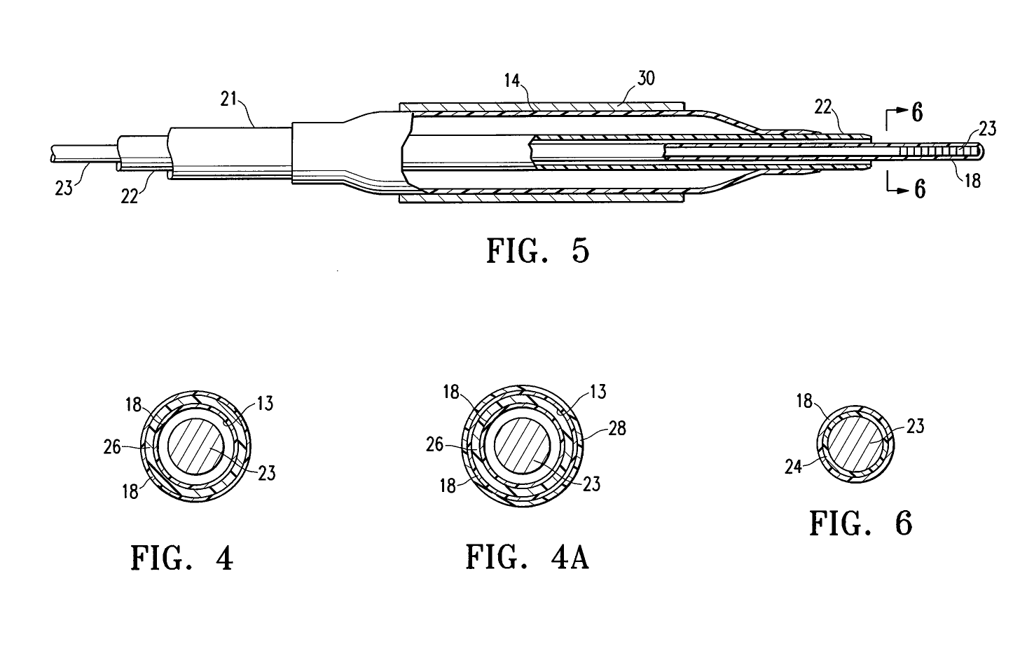

The distal tip section 26 of the shaft 11, formed by the distal end of the inner tubular member 22 and/or by a soft distal tip member secured to the distal end of the inner tubular member 22 and/or balloon proximal skirt, has the lubricious coating 18 on the outer and the inner surface thereof, as best shown in FIG. 4, illustrating a transverse cross section of the distal tip section 26 of the catheter 10 of FIG. 1, taken along line 4-4. However, in alternative embodiments, the lubricious coating 18 is located on just the outer or just the inner surface of the distal tip section 26. FIG. 4a illustrates an alternative embodiment in which the lubricious coating 18 on the outer surface of the distal tip section 26 is further coated with a second, different lubricious coating, which in the embodiment of FIG. 4a is the same lubricious coating 28 that is on the balloon. The lubricious coating 18 is sufficiently durable to remain on the distal tip section 26 during assembly of the catheter 10, so that in one embodiment, the lubricious coating 18 is provided on the distal tip section 26 of the catheter prior to assembly and processing of the catheter 10, for example by dip coating or wiping on a distal tip member before it is attached to the inner member and/or balloon. After assembly of the catheter, the second lubricious coating 28 is applied on the balloon 14 and tip 26. The undercoat of lubricious coating 18 of the invention on the distal tip 26 is provided to minimize variations, and enhance the durability of the lubricity of the distal tip 26 of the fully assembled catheter, which improves the ability of the catheter to cross tight stenoses in the patient's body lumen. In a presently preferred embodiment, the hydrophilic coating applied to the distal tip before it is attached to the catheter is the interlocking network lubricious coating 18 discussed in more detail below, although in alternative embodiments a variety of suitable hydrophilic lubricious coatings including PEO or PVP based coatings can be applied to the distal tip before it is attached to the catheter in accordance with a method of the invention.

Although illustrated in the embodiment of FIG. 1 on the outer tubular member 21, inner tubular member 22, and distal tip section 26 of the catheter 10, it should be understood that the coating 18 can alternatively be applied to fewer areas of the catheter 10 such as just the outer tubular member 21, or to different areas of the catheter 10. Thus, the lubricious coating 18 of the invention can be applied to a variety of suitable locations on the catheter 10. Additionally, the lubricious coating 18 can be applied to a variety of suitable alternative medical devices. For example, FIG. 5 illustrates the lubricious coating 18 on guidewire 23. Guidewire 23 comprises a metallic core and coiled wire distal tip, and the coating 18 is preferably along at least a distal section of the guidewire including the floppy distal tip. Guidewire 23 having the lubricious coating 18 of the invention thereon preferably advances and retracts with very low friction force within the guidewire lumen of a catheter.

As best shown in FIG. 6, illustrating a transverse cross section of the guidewire of FIG. 5, in the embodiment of FIG. 5 the guidewire has a polymer layer 24 on an outer surface of the metallic core such that the lubricious coating 18 is on an outer surface of the guidewire polymer layer 24. In one embodiment, the polymer layer 24 is a polyurethane coating or layer on a stainless steel or NiTi core wire of the guidewire, although the polymer layer 24 can be formed of a variety of polymers including polyolefin, copolyamides, copolyesters or filled polyurethane. Fillers such as tungsten, barium, and bismuth and their compounds in general can be added to enhance radiopacity.

The lubricious coating 18, on catheter 10 and/or guidewire 23, comprises the cured reaction product of a solution mixture comprising a multifunctional monomer or polymer network-forming compound; a combination of long chain and short chain hydrophilic compounds; one or more first cross-linkers for cross-linking the multifunctional monomer or polymer, which preferentially cross-links the multifunctional monomer or polymer relative to the hydrophilic compounds; and one or more second cross-linkers, different than the first cross-linkers, for cross-linking the hydrophilic compounds, which preferentially cross-links the hydrophilic compounds relative to the multifunctional monomer or polymer. The resulting cured coating on the medical device is a network of the hydrophilic compounds cross-linked to one another and interlocked with a network of the cross-linked polymerized multifunctional monomer or polymer.

The multifunctional network-forming compound is preferably a triacrylate oligomer such as a high molecular weight ethoxylated trimethylol propane triacrylate (ETMPTA) (e.g., PHOTOMER® 4158, available from Cognis). The ETMPTA oligomer polymerizes and cross-links during curing to form a network of cross-linked ETMPTA. Alternative cross-linkable polymers (formed from alternative multifunctional monomers or polymers) for forming an interlocking network with the hydrophilic compound include urethane, epoxy, polyester acrylates, and unsaturated polyesters, although a triacrylate, and particularly ETMPTA, is preferred due to its enhanced hydrophilic property, and compatibility with common solvents for good manufacturability. Less preferred is a methacrylate due to the slow reaction and sensitivity to oxygen.

Preferred cross-linkers are photosensitive molecules (photo cross-linkers). Specifically, in the embodiment in which the multifunctional oligomer is a triacrylate, the solution mixture preferably includes mixed first photoinitiators including benozophenone, and a benzil dimethyl ketal such as 2,2-dimethoxy-2-phenyl acetophenone (PHOTOMER® 51, available from Cognis) for photocuring the triacrylate. A variety of mixed first photoinitiators are typically provided, which work by different mechanisms to initiate polymerization and cross-linking of the triacrylate (and acrylates in general) as is generally known. For example, upon irradiation, PHOTOMER®51 undergoes a unimolecular bond cleavage to yield free radicals, while the benezophenone undergoes a bimolecular reaction in the presence of alcohol in which hydrogen abstraction creates hydroxyl (or ketal-type) radicals. However, a variety of suitable first photo cross-linkers can be used which preferentially cross-link the multifunctional polymerized monomer or polymer (e.g., triacrylate oligomer). For example, alternative photoinitiators for cross-linking the triacrylate include 1-hydroxy-cyclohexyl-phenyl-ketone, and 2-hydroxy-2-methyl-1-phenyl-1-propanone, although the preferred photoinitiators provide superior manufacturability due at least in part to good solubility. Ultraviolet, as opposed to visible light, photoinitiation is preferred for faster curing time.

Presently preferred hydrophilic compounds comprise polyvinylpyrrolidone (PVP, (poly(N-vinyl-2-pyrrolidone)), which, when in combination with the second photo cross-linker such as a diazidostilbene (DAS) or derivative thereof, cross-link during curing to form a network of cross-linked PVP. In accordance with the present invention a combination of PVP K90 and PVP K30 may be employed wherein the K number is related to the molecular weight of the PVP. PVP K90 has a molecular weight greater than 1,000,000 g/mole while PVP K30 has a molecular weight of about 44,000 g/mole. PVP K30 and PVP K90 are available from ISP chemicals. Alternatively, POLYOX WSR N-10 (M_{w}=100,000 g/mole) or POLYOX WSR N-80 (M_{w}=200,000 g/mole) can be substituted for the PVP K30. All such polymers are readily mixable since they are all water soluble. The addition of a short chain polymer has the additional benefits of reducing the overall viscosity of the formulation which would tend to benefit a brush coating process to the extent that the creation of bubble defects is minimized especially for balloon catheter applications. A presently preferred diazidostilbene for preferentially cross-linking the PVPs is 4,4′-diazido-2,2′-stilbene disulfonic acid disodium salt. Other possible diazido based second photo cross-linkers that could be used include diazidostilbene derivatives including those set forth in U.S. Pat. No. 5,041,570, the Summary and Detailed Description of the Invention of which are hereby incorporated by reference. Upon irradiation, DAS (a photo cross-linking agent) forms a highly reactive intermediate nitrene group on both ends, and then the nitrene groups on the DAS will react with PVPs to form the cross-linked network of PVPs. In accordance with the invention, the DAS preferentially cross-links the PVPs relative to the multifunctional monomer or polymer network-forming compound (e.g., the triacrylate). That is, the DAS cross-links PVP polymer chains together, substantially without cross-linking the polymer chains of the multifunctional polymerized monomer or polymer. Similarly, the first photo cross-linkers are not expected to cross-link the hydrophilic compounds (PVPs) of the coating of the invention. Additionally, curing the coating does not cross-link, graft or otherwise chemically bond the hydrophilic compounds to the polymerized monomer or polymer, or to the substrate. Thus, although a variety of hydrophilic compounds are well known for use in lubricious coatings for medical devices, in the coating of the invention the hydrophilic compounds have a specific initiator which can be added to the solution mixture to preferentially cross-link the hydrophilic compounds to one another to a desired degree. Alternative hydrophilic compound-second photo cross-linker combinations that can be used in the coating of the invention include the combination of polyethylene glycol diacrylates (PEGDA) and the photoinitiator 2,2-dimethoxy-2-phenylacetophenone.

The amount of the second cross-linkers provided in the solution mixture relative to the amount of the hydrophilic compounds, and the duration of the curing is sufficient to form a three dimensional cross-linked network of the hydrophilic compounds, although the hydrophilic compounds are cross-linked to a greater or less degree depending on the desired performance characteristics of the lubricious coating 18. The control provided by the invention over the cross-linking of the hydrophilic compounds facilitates creating a desired lubricity and durability which can be tailored for different applications. Thus, PVP that is part of the network in lubricious coating 18 has a greater or lesser degree of cross-linking. Additionally, some noncross-linked hydrophilic compounds (i.e., PVP that is not cross-linked and thus not part of the network) or a noncross-linked secondary hydrophilic compound such as PEO are present in the lubricious coating in some embodiments, for enhanced lubricity at the potential expense of durability. Specifically, network lubricious coatings in which durability and not lubricity was at issue would cross-link the hydrophilic compounds to a greater degree to maximize the durability of the coating at the expense of the lubricity, which may be acceptable in some applications. Additionally, because the cross-linking of the hydrophilic compounds is more readily controllable in the lubricious coating of the invention, the amount of cross-linking caused by initially photo-curing the coating on the device can be tailored to compensate for any additional cross-linking that may occur later, as for example when sterilizing the coated device by e-beam or EtO sterilization causes further cross-linking of the coating. In one embodiment, the coated device is e-beam sterilized, and the method of coating the device involves (UV) curing the coating on the device for a relatively short duration which is insufficient to cross-link the compounds to the desired degree (e.g., as determined by performance testing of the coated medical device), and subsequently e-beam sterilizing the coated device such that the compounds further cross-link to the desired degree. Similarly, the amount of photo cross-linkers in the coating can be limited to control the amount of cross-linking caused by the photo-curing.

The solution mixture is formed by combining the multifunctional monomer or polymer, the short chain and long chain hydrophilic compounds, one or more first cross-linkers, and one or more second cross-linkers together in a single solution (the compounds typically having been first dissolved in a suitable solvent before combining to form the single solution). The solution mixture is then applied to the surface of the catheter shaft 11 and/or guidewire 23, and it can be applied to the device using a variety of suitable methods including dipping, spraying, wiping the solution on the surface of the catheter or guidewire, or drawing the solution through the guidewire lumen 13 of the catheter. The coating is then typically dried on the device before the curing, and the resulting cured coating has the substantially uniform composition provided by the interlocked networks in a single layer. In one embodiment, an adhesion promoting primer is first coated onto the device and cured, and then the lubricious coating solution mixture is applied onto the cured primer. The cured coating 18 has to be hydrated to render it lubricious for use in a medical procedure. The water induction time, i.e., the time required to hydrate the coating, varies depending on the coating formulation and more specifically, the amount of short chain hydrophilic polymers in the composition. Thus, the terminology “lubricious coating” as used herein should be understood to refer to the finished coating on the device, either before or after the hydrophilic compounds are hydrated to render the coating lubricious for use.

In one embodiment, the solution mixture includes an adhesion promoter comprising an acid functionalized acrylate which adheres to a surface of the medical device to improve adhesion of the lubricious coating 18 on the medical device. The preferred adhesion promoter bonds to the surface of the substrate (e.g., the polymer surface of the catheter shaft or the guidewire) and also cross-links to the multifunctional polymerized monomer or polymer. Thus, the first initiators preferably cross-link the adhesion promoter, such that the adhesion promoter is cross-linked to itself and to the cross-linked polymerized multifunctional monomer or polymer in the cured lubricious coating. A presently preferred adhesion promoter is PHOTOMER® 4173, an acid functionalized monoacrylate from Cognis, which bonds to a polymeric (and particularly a polyurethane) substrate layer. Alternative adhesion promoters which could be used include the acid functionalized acrylates PHOTOMER® 4703 and 4846 from Cognis. The adhesion promoter is generally about 0.2% to about 20%, more specifically about 1% to about 2%, by weight of the solution mixture. A reactive primer layer on the device, such as these acid functionalized adhesion promoters (plus a photoinitiator) or other primer compounds such as a urethane acrylate, could additionally or alternatively be used to improve adhesion. With or without the adhesion promoter, the coating 18 of the invention adheres to the surface of the device without requiring that the hydrophilic compounds are functionalized or otherwise made to reactively chemically bond to a matrix or substrate polymer.

In one embodiment, the solution mixture includes a secondary hydrophilic compound such as polyethylene oxide (PEO) which is different than the network forming hydrophilic compounds (e.g., PVPs). The secondary hydrophilic compound is substantially noncross-linked in the lubricious coating. Thus, an initiator which preferentially cross-links the secondary hydrophilic compound is not included in the solution mixture, and curing the coating produces relatively little or no cross-linking of the secondary hydrophilic compound. As a result of being substantially noncross-linked, the secondary hydrophilic compound preferably provides a coating which is, at least initially, more lubricious and/or which has a decreased water induction time (i.e., a quicker response to a hydration procedure). For example, a substantially noncross-linked hydrophilic compound such as polyethylene oxide (PEO) in the coating hydrates relatively quickly. Specifically, combining the primary hydrophilic compounds such as PVPs with the secondary hydrophilic compound such as PEO or polyacrylamide provides a coating that preferably has an improved, fast water induction time after sterilization by e-beam or EtO treatment. Noncross-linked PEO or polyacrylamide preferably compensates for an increase in water induction time of the lubricious coating due to both e-beam and EtO sterilization. A variety of suitable hydrophilic compounds can be used as the secondary hydrophilic compound including PEO, polyacrylamide-co-acrylic acid and polyacrylamide. In one embodiment, a relatively small amount of the secondary hydrophilic compound is present in the coating. For example, in one embodiment, the secondary hydrophilic compound is only about 5% by weight of the amount of PVP in the lubricious coating.

In one embodiment, the solution mixture includes a dissolvable ionic compound (i.e., a salt) such as sodium chloride, and the resulting cured lubricious coating has the salt contained (dissolvably) therein at least prior to the hydration procedure used to hydrate the coating for use. The water induction time is believed to decreased relative to the coating without the salt as a result of the presence of the salt in the cured coating.

In one embodiment, the cured lubricious coating has a therapeutic or diagnostic agent. For example, an agent added to the solution mixture is releasably contained in the cured coating such that as the cured coating swells (hydrates) during use, the agent will elute therefrom. The cured lubricious coating can be provided with a variety of agents. Anti-platelet agents, anti-thrombogenic agents, anti-coagulant agents, anti-inflammatory agents, vasodilator agents, and the like are particularly preferred for adding to the lubricious coating on the balloon 14, outer member 21, guidewire 23, and/or within the guidewire lumen 13 of the catheter shaft 11. A relatively small molecule agent such as aspirin (acetylsalicyclic acid; acetolsal) is particularly desirable in the lubricious coating because its relatively quick elution time from the lubricious coating provides a concentrated quick dose of the aspirin during the initial introduction and advancement of the catheter and/or guidewire in the patient's body lumen. Although controlled, longer term elution of agents from medical device coatings is a goal of many of prior art coatings, relatively quick, uncontrolled elution of the aspirin from the lubricious coating of the invention is desirable. The concentrated release of the aspirin from the lubricious coating upon hydration of the coating provides an anti-platelet affect during positioning of the catheter in the body lumen, which further reduces guidewire hang-up in the catheter guidewire lumen. Although aspirin has a small molecular weight (e.g., 180 g/mol), alternative agents with larger molecular weights than aspirin can alternatively be used in a coating of the invention, such as Hirudin (about 7,000 g/mol) or Heparin (about 12,000 to about 15,000 g/mol).

The lubricious coating of the invention can be provided with a variety of suitable agents (small or large molecule agents) including anti-restenosis agents, and anti-inflammatory, anti-coagulating, or pro-healing drugs. The agent is typically provided by adding it into the solution mixture prior to application onto the device, which is a preferred method due to the good manufacturability, control over the amount and location of the agent on the device, and minimal disruption of the lubricity of the coating. Less preferred methods include swelling the cured coating on the device with a solution of the agent prior to use.

In the embodiment illustrated in FIG. 1, the coating 28 on the balloon 14 is different than the lubricious coating 18 on the shaft. For example, the coating 28 on the balloon may be a lubricious coating which has less lubricity or may contain a different therapeutic agent than the coating on the shaft. In alternative embodiments as discussed above, the same lubricious coating 18 on the shaft 11 is provided on the balloon 14.

In one embodiment, a lubricious coating 28 on the balloon 14 has a relatively short water induction time (hydrates quickly) and includes an anti-restenosis agent such as everolimus or zotarolimus for treating artery disease and/or preventing restenosis. The agent is well preserved in the agent delivery lubricious coating 28 before balloon inflation, and since the water up-take by the agent delivery lubricious coating 28 occurs quickly, the agent is released immediately as the balloon 14 is inflated, for providing a sufficient dose of the agent at the desired site. Typically, the balloon prior to inflation is folded and thus protects some of the coating within the folds as the catheter is first hydrated and advanced within the blood vessel. In one embodiment, the agent delivery lubricious coating 28 on the balloon is the embodiment of the interlocking network lubricious coating described above having the noncross-linked secondary hydrophilic compound added thereto which provides a quick water induction (e.g., noncross-linked PEO in the interlocking network of cross-linked PVPs and cross-linked triacrylate). As discussed above, the agent is preferably added to the solution mixture of the lubricious coating prior to coating of the balloon. The balloon having the agent delivery lubricious coating thereon is then folded or otherwise configured into a low profile configuration for advancement within the patient's body lumen.

In one embodiment, coating 28 on the balloon is a less lubricious coating than the lubricious coating 18 on the shaft, to prevent or inhibit the inflated balloon from slipping out of the desired treatment location in the patient's body lumen (commonly referred to as “watermelon seeding”). There are a number of alternate approaches to making the coating 28 on the balloon as a less lubricious coating than the lubricious coating 18 on the shaft. For example, a more dilute concentration solution of the same ingredients can be applied on the balloon after the same or more concentrated solution is applied over the shaft and balloon. As another example, a coating comprised of the solution incorporating one hydrophilic polymer (for example PEO) can be applied on the balloon, while a coating comprised of the solution incorporating different hydrophilic polymers (for example PVPs) can be applied on the shaft. As another example, the lubricious coating 28 can comprise the reaction product of a solution mixture of a binding multifunctional oligomer (or monomer or higher molecular weight polymer), a photo cross-linker for cross-linking the binding oligomer, and a hydrophilic compound without a photo cross-linker for preferentially cross-linking the hydrophilic compound of the less lubricious coating. The coating 28 on the balloon can thus be formed of the same component compounds as the coating 18 on the shaft but without the second photo cross-linkers, to result in a less lubricious coating. Although coating 28 is illustrated extending along the entire length of the balloon from the proximal to the distal ends of the balloon, it should be understood that in alternative embodiments, the coating 28 can extend along a shorter length of the balloon or beyond the ends of the balloon.

The following example illustrates a solution mixture for a lubricious coating 18 of the invention. In addition to the specific formulation (with the amount of each component expressed as a weight percent of the solution mixture) used in the following example, the Table also gives example solution weight percent ranges for the components which can be used in making coatings of the invention.

TABLE

| | Specific Weight % | General Weight % |
|---|---|---|
| Chemical | (Formulation A) | Range Formulations |
| Ethanol | 79.63 | about 60 to about 80 |
| Isopropanol (IPA) | 5.53 | about 2 to about 10 |
| Water | 5.53 | about 2 to about 10 |
| PVP K-90 and PVP K-30 | 6.30 | about 2 to about 10 |
| PEO | 0 | about 0 to about 10 |
| PHOTOMER ® 4173 | 1.02 | about 0 to about 5 |
| PHOTOMER ® 4158 | 1.89 | about 1 to about 5 |
| PHOTOMER ® 51 | 0.019 | about 0.01 to about 0.05 |
| Benzophenone | 0.019 | about 0.01 to about 0.05 |
| 4,4′-diazido- | 0.063 | about 0.01 to about 0.25 |
| 2,2-stilbenedisulfonic | | |
| acid disodium salt hydrate | | |

A solution mixture of formulation A listed in the Table was applied by dip coating onto a guidewire which had a metallic core wire covered by a polymer layer of a tungsten filled polyurethane polymer. In a testing procedure in which the coated guidewire is repeatedly advanced and retracted within a guidewire lumen of a catheter inner tubular member having an HDPE inner surface (the inner tubular member being filled with sterile water and kept at 37° C. with a 1.25″ loop), the resulting frictional force caused by the movement of the coated guidewire in the guidewire lumen remained low after multiple cycles, up to 1000 cycles and after twenty four hours. The frictional force after multiple cycles was lower when compared to a guidewire otherwise the same but coated with a lubricious coating of PEO in a cross-linked acrylate (i.e., a solution mixture of isopropanol, water, PEO, trimethylolpropyl triacrylate (TMPTA), hydroxycyclohexyl phenyl ketone and benzophenone, wherein the PEO was a POLYOX WSR N12K and was about 1.6 weight percent of the solution mixture). For example, after thirty cycles, the friction force during pulling or pushing of the guidewire coated with formulation A set forth in the above Table was about 5 grams compared to about 35 to 55 grams for the comparison guidewire.

The dimensions of catheter 10 are determined largely by the size of the balloon and guidewire to be employed, the catheter type, and the size of the artery or other body lumen through which the catheter must pass or the size of the stent being delivered. Typically, the outer tubular member 21 has an outer diameter of about 0.025 to about 0.04 inch (0.064 to 0.10 cm), usually about 0.037 inch (0.094 cm), and the wall thickness of the outer tubular member 21 can vary from about 0.002 to about 0.008 inch (0.0051 to 0.02 cm), typically about 0.003 to 0.005 inch (0.0076 to 0.013 cm). The inner tubular member 22 typically has an inner diameter of about 0.01 to about 0.018 inch (0.025 to 0.046 cm), usually about 0.016 inch (0.04 cm), and a wall thickness of about 0.004 to about 0.008 inch (0.01 to 0.02 cm). The overall length of the catheter 10 may range from about 100 to about 150 cm, and is typically about 143 cm. Preferably, balloon 14 has a length about 0.8 cm to about 6 cm, and an inflated working diameter of about 2 mm to about 10 mm. The guidewire 23 typically has length of about 190 to about 300 cm, and an outer diameter of about 0.010 to about 0.035 inch.

The various catheter components may be joined using conventional bonding methods such as by fusion bonding or use of adhesives. Although the shaft 11 is illustrated as having an inner and outer tubular member, a variety of suitable shaft configurations may be used including a dual lumen extruded shaft having a side-by-side lumens extruded therein. Additionally, although the embodiment illustrated in FIG. 1 is an over-the-wire type balloon catheter having a guidewire lumen extending the full length of the catheter, it should be understood that the coating 18 of the invention can be used with a variety of suitable catheters including guiding catheters having a device lumen configured for delivering catheters or other devices, or rapid-exchange type balloon catheters having a guidewire proximal port spaced distally from the proximal end of the catheter shaft.

While the present invention is described herein in terms of certain preferred embodiments, those skilled in the art will recognize that various modifications and improvements may be made to the invention without departing from the scope thereof. For example, although discussed primarily in terms of a coating on a catheter shaft or guidewire, it should be understood that the lubricious coating 18 of the invention can be provided on a variety of medical devices, and is particularly suitable for use on surfaces encountering significant rubbing or abrasive forces during use or assembly and processing. Moreover, although individual features of one embodiment of the invention may be discussed herein or shown in the drawings of the one embodiment and not in other embodiments, it should be apparent that individual features of one embodiment may be combined with one or more features of another embodiment or features from a plurality of embodiments.

What is claimed:

1. A medical device having at least a section with a lubricious coating that comprises the cured reaction product of a solution mixture applied to the device, the solution mixture, comprising:
a) a multifunctional monomer or polymer network-forming compound;
b) a short chain hydrophilic compound;
c) a long chain hydrophilic compound;
d) one or more first cross-linkers for cross-linking the multifunctional monomer or polymer, which preferentially cross-links the multifunctional monomer or polymer relative to the hydrophilic compounds; and
e) one or more second cross-linkers, different than the first cross-linkers, for cross-linking the hydrophilic compounds, which preferentially cross-links the hydrophilic compounds relative to the multifunctional monomer or polymer, such that the cured reaction product on the medical device is a network of the hydrophilic compounds cross-linked to one another and interlocked with a polymerized network of the monomer or polymer.

2. The method of claim 1, wherein the lubricious coating is cured using a radiation source.

3. The method of claim 1, wherein the lubricious coating is cured using an ultraviolet source.

4. The method of claim 1, wherein the lubricious coating is cured using an electron beam source.

5. The device of claim 1, wherein the network-forming compound is a triacrylate.

6. The device of claim 1, wherein the network-forming compound is an ethoxylated trimethylol propane triacrylate oligomer.

7. The device of claim 1, wherein the hydrophilic compounds comprise polyvinylpyrrolidone.

8. The device of claim 7, wherein the short claim hydrophilic compound comprises PVP K30.

9. The device of claim 7, wherein the long claim hydrophilic compound comprises PVP K90.

10. The device of claim 1, wherein the hydrophilic compound network is not chemically bonded to the polymerized monomer or polymer network.

11. The device of claim 1, wherein the solution mixture includes an adhesion promoter comprising an acid functionalized acrylate which adheres to a surface of the medical device.

12. The device of claim 11, wherein the first cross-linkers cross-link the adhesion promoter, such that the adhesion promoter is cross-linked to itself and to the polymerized monomer or polymer in the lubricious coating.

13. The device of claim 11, wherein the device is a metal guidewire with a polymeric outer layer, and the coating is adhered directly to the polymeric outer layer of the guidewire without a reactive primer between the polymer layer and the coating.

14. The device of claim 11, wherein the device is a metal guidewire, and the coating is adhered directly to the metal guidewire without a reactive primer between the polymer layer and the coating.

15. The device of claim 11, wherein the device is a metal guidewire with a polymeric outer layer with a primer layer of an adhesion promoter, and the lubricious coating is adhered to the primer layer.

16. The device of claim 11, wherein the device is a guide catheter with a polymeric outer layer, and the coating is adhered directly to the polymeric outer layer of the guide without a reactive primer between the polymer layer and the coating.

17. The device of claim 11, wherein the device is a guide catheter with a polymeric outer layer, and the coating is adhered to the polymeric outer layer of the guide with a reactive primer between the polymer layer and the coating.

18. The device of claim 1, wherein the device is a guidewire having a metal surface with a primer layer of an adhesion promoter, and the lubricious coating is adhered to the primer layer.

19. The device of claim 1, wherein the device is a balloon catheter having an elongated catheter shaft and a balloon on a distal shaft section, with the lubricious coating on at least a section of the shaft, and on at least a section of the balloon.

20. The device of claim 1, wherein the device is a guide catheter with a polymeric outer layer, and the coating is adhered directly to the polymeric outer layer of the guide without a reactive primer between the polymer layer and the coating.

21. The device of claim 1, wherein the device is a guide catheter with a polymeric outer layer, and the coating is adhered to the polymeric outer layer of the guide with a reactive primer between the polymer layer and the coating.

22. The device of claim 1, wherein the first and/or second cross-linkers are photo cross-linkers such that the coating is photo-cured.

23. The device of claim 22, wherein the second photo cross-linker is a diazido compound.

24. The device of claim 23, wherein the diazido compound is a diazidostilbene or a diazidostilbene derivative.

25. The device of claim 22, wherein the first photo cross-linkers are benzophenone and benzil dimethyl ketal.

26. The device of claim 1, wherein the coating includes a secondary hydrophilic compound which is different than the cross-linked hydrophilic compounds and which is substantially noncross-linked in the lubricious coating.

27. The device of claim 1, wherein the solution mixture includes a salt, and the salt is dissolvably contained in the cured coating at least prior to hydration of the coating.

28. The device of claim 1, wherein the solution mixture includes a therapeutic agent, such that the networks form in the presence of the therapeutic agent, and the therapeutic agent is releasably contained in the lubricious coating.

29. The device of claim 28, wherein the therapeutic agent is a relatively small molecule agent which elutes relatively quickly in a concentrated release from the lubricious coating upon hydration of the coating.

30. The device of claim 27, wherein the therapeutic agent is acetylsalicyclic acid.

31. A method of providing a lubricious coating for a medical device, comprising:
a) preparing a solution mixture of a multifunctional monomer or polymer, a relatively short chain hydrophilic compound, a relatively long chain hydrophilic compound, one or more first cross-linkers for cross-linking the monomer or polymer, which preferentially cross-links the monomer or polymer relative to the hydrophilic compounds, and one or more second cross-linkers, different than the first cross-linkers, for cross-linking the hydrophilic compounds, which preferentially cross-links the hydrophilic compounds relative to the monomer or polymer; and
b) applying a coating of the solution mixture onto a surface of at least a section of the medical device and curing the coating such that the resulting lubricious coating is a network of the hydrophilic compound cross-linked to itself and interlocked with a polymerized network of the monomer or polymer.

32. The method of claim 31, wherein the hydrophilic coating is cured using a radiation source.

33. The method of claim 32, wherein the radiation source is an ultraviolet light.

34. The method of claim 31, wherein an amount of the second cross-linkers is limited so that curing the coating cross-links the hydrophilic compound to a lesser degree than desired, and including e-beam or EtO sterilizing the device after claim 22 to further cross-link the hydrophilic compound to a desired degree.

35. The method of claim 31, wherein a duration of the curing is limited so that curing the coating cross-links the hydrophilic compound to a lesser degree than desired, and including e-beam or EtO sterilizing the device after b) to further cross-link the hydrophilic compound to a desired degree.

36. The method of claim 31, wherein curing the coating does not chemically bond the hydrophilic compounds to the monomer or polymer.

37. The method of claim 31, wherein the solution mixture includes an adhesion promoter comprising an acid functionalized acrylate which adheres to a surface of the medical device.

38. The method of claim 31, wherein b) includes cross-linking the adhesion promoter to itself and to the multifunctional polymerized monomer or polymer.

39. The method of claim 31, wherein the solution mixture includes a secondary hydrophilic compound without an initiator which would preferentially cross-link the secondary hydrophilic compound, and wherein curing the coating does not cross-link the secondary hydrophilic compound, such that the secondary hydrophilic compound is noncross-linked in the lubricious coating.

40. The method of claim 31, wherein the device is a balloon catheter having an elongated catheter shaft with a distal tip member and a balloon on a distal shaft section, and the solution mixture is applied to an inner and/or an outer surface of the distal tip member before the distal tip member is bonded to the catheter shaft.

41. The method of claim 31, wherein the lubricious coating is cured using an ultraviolet source.

42. The method of claim 31, wherein the lubricious coating is cured using an electron beam source.
